(19) United States
(12) Patent Application Publication
(10) Pub. No.: US 2011/0065791 A1
(43) Pub. Date: Mar. 17, 2011

(54) SYSTEM FOR AROMA RELEASE

(76) Inventors: Albert Thijs Poortinga, Apeldoorn (NL); Paul Bastiaan van Seeventer, Meppel (NL); Mathijs Hendrikus Johannes Martens, Tolkamer (NL); Derrick Adriaan Johannes Kirpestein, Vollenhove (NL)

(21) Appl. No.: 12/919,127

(22) PCT Filed: Feb. 25, 2009
(86) PCT No.: PCT/NL2009/050085

§ 371 Date: Nov. 19, 2010

(30) Foreign Application Priority Data
Feb. 25, 2008 (NL) 2001320

(51) Int. Cl. A23L 1/22; A61K 8/37; A61Q 90/00; A61Q 19/10; C11D 17/00; B01J 13/04

(52) U.S. Cl. 514/552; 514/546; 426/519; 426/650; 510/130; 510/438; 510/218; 264/4.1; 426/534

(57) ABSTRACT

The invention concerns a process for preparing particles, which particles contain one or more spaces in which a gas phase is present which comprises at least one active ingredient, in particular at least one aroma, flavor or precursor for an aroma or flavor, and which space or spaces are at least substantially surrounded by an enveloping phase which at ambient temperature is at least substantially solid and at least substantially impermeable to the active ingredient, comprising allowing the gaseous active ingredient to migrate from or through the enveloping phase into the space or spaces at a temperature at which the enveloping phase is permeable to the gaseous active ingredient; and then cooling the particles to a temperature at which the enveloping phase of the particles is at least substantially impermeable to the active ingredient in the particles.

The invention relates to the preparation of particles with a gas phase containing an active ingredient, such as an aromatic substance. The invention further relates to particles obtainable by the invention.

Odor is an important property of foodstuffs. The odor of products can be boosted by adding aromatic substances (aromas). However, this entails the disadvantage that the taste of products may be adversely affected by the addition of extra aromas, for instance becomes too strong. For this reason, there is a need for systems that release volatile aromas in products without this having an effect on the taste that is experienced as negative by consumers, or at least where such an effect is smaller. In the literature, a number of systems are described which serve to augment the aroma of products.

U.S. Pat. No. 4,520,033 describes a process for the preparation of foamed aroma capsules. The process concerns the mixing of an aqueous liquid containing aromatic constituents, such as coffee or tea distillate, with a water-soluble powder to form a basic mixture, foaming the basic mixture, and coating droplets of the foamed basic mixture with a powder. Forming the foam serves to lower the density, so that the particles float on water.

WO 96/07333A1 describes a co-extrusion process for preparing capsules containing an aroma dissolved in an edible oil. In the oil, furthermore, a gas is dissolved. Upon dissolution of the capsules, the intention is for gas bubbles to be formed by the dissolved gas, whereby the aromas are to be released. The process described is rather complex. Further, the aroma must first evaporate from the oil phase, so that release is relatively slow. Moreover, the presence of the oil may lead to a visible oil film on a product in which the capsules are dissolved, which may be experienced as undesirable. In U.S. Pat. No. 5,496,574 it is proposed to use hydrolyzed oil to obviate this last-mentioned disadvantage. Hydrolyzed oil, however, may contain undesirable flavoring or aromatic substances, and there are indications that the aroma-containing droplets that are finely divided in the product strongly retard aroma release.

US 2002/0119235A1 concerns coffee aroma compositions of a water-insoluble, liquid carrier medium and coffee aromas. In particular, powders are described in which such composition is encapsulated. In the use of such a composition, there is a real chance that upon dissolution of the powder during use also a large part of the aroma is dissolved, resulting in limited or retarded availability as aroma. Also, the necessity of a liquid additive (the carrier medium) may be unwanted.

EP-A1522 223 describes aroma particles which comprise a water soluble coffee matrix and an encapsulated liquid phase, which liquid phase contains an aromatizing composition. The particles can be prepared by a process whereby coffee extract is foamed and then mixed with an aromatizing composition. From the mixture, droplets are made which are mixed with milled coffee powder, after which the mixture is dried and the obtained particles are separated from excess coffee powder. The required drying step can lead to loss of aromas.

It is an object of the invention to provide a new process for preparing particles which contain an active ingredient, in particular an aroma. In particular, it is an object to provide a process that does not have one or more of the above-mentioned disadvantages or at least exhibits such disadvantage to a lesser extent.

It is in particular an object of the invention to provide a process that is relatively simple to apply, also on an industrial scale, and/or with an improved release of a volatile active ingredient, in particular an aroma.

It is furthermore an object of the invention to provide particles with an active ingredient, such as an aroma.

It has now been found that it is possible to prepare particles with a gas phase containing a volatile active ingredient, such as an aroma, which upon release from the particles brings about a desired effect, such as a particular olfactory sensation. In particular, it has been found that this can be realized while an undesired effect, such as a negative taste effect, does not occur or at least does not occur to an unacceptable extent.

Accordingly, the invention relates to a process for preparing particles, which particles contain one or more spaces in which a gas phase is present which comprises at least one active ingredient, in particular at least one aroma, flavor or precursor for an aroma or flavor, and which space or spaces are at least substantially surrounded by an enveloping phase which at ambient temperature is at least substantially solid and at least substantially impermeable to the active ingredient, comprising allowing the gaseous active ingredient to migrate from or through the enveloping phase into the space or spaces at a temperature at which the enveloping phase is permeable to the gaseous active ingredient; and then cooling the particles to a temperature at which the enveloping phase of the particles is at least substantially impermeable to the active ingredient in the particles.

In an embodiment, the present invention concerns a process for preparing particles, which particles contain one or more spaces in which a gas phase is present which comprises at least one active ingredient, in particular at least one aroma, flavor, or a precursor for an aroma or flavor, and which space or spaces are at least substantially surrounded by an enveloping phase which at ambient temperature is at least substantially solid and at least substantially impermeable to the active ingredient,

comprising the mixing of particles, which contain one or more spaces, with a gas which comprises the gaseous active ingredient, at a temperature at which the enveloping phase is permeable to the active ingredient, whereby gaseous active ingredient migrates into the spaces of the particles, and then cooling the particles to a temperature at which the enveloping phase of the particles is at least substantially impermeable to the active ingredient in the particles.

In an embodiment, the invention concerns a process for preparing particles, which particles contain one or more spaces in which a gas phase is present which comprises at least one active ingredient, in particular at least one aroma, flavor or precursor for an aroma or flavor, and which space or spaces are at least substantially surrounded by an enveloping phase which at ambient temperature is at least substantially solid and at least substantially impermeable to the active ingredient, wherein the enveloping phase of the particles which are provided with the active ingredient comprises a precursor for the active ingredient, which precursor upon heating is converted into the gaseous active ingredient, comprising the heating of the particles of which the enveloping phase contains a precursor, thereby forming the gaseous active ingredient; allowing the gaseous active ingredient to migrate from the enveloping phase into the space or spaces; and then cooling the particles to a temperature at which the enveloping phase of the particles is at least substantially impermeable to the active ingredient. In a preferred embodiment, the particles are heated while mixing the particles with a gas.

The invention furthermore relates to particles obtainable by a process according to the invention.

The invention provides particles where the enveloping phase is at least substantially free of the active ingredient. In certain cases, it is possible that a small part of the active ingredient is present (dissolved or dispersed) in the enveloping material, such as 25 wt. % or less, in particular 10 wt. % or less, more in particular 5 wt. % or less.

The invention furthermore relates to a spray-dried powder, which powder comprises particles, which particles contain one or more spaces in which a gas phase is present which comprises at least one gaseous active ingredient, preferably an active ingredient selected from the group of aromas, flavors, aroma precursors, flavor precursors, and oxidation-sensitive active ingredients.

It is surprising that it is possible to introduce active ingredient at least substantially into particles in a suitable amount to bring about a desired effect, such as a particular olfactory sensation. Without being bound by theory, the inventors suspect that at least in a process in which the mixing is done at an elevated temperature, the amount of volatile active ingredient in the particles is relatively high, and possibly at or near the saturation level at the temperature after cooling, so that the gas phase in the particles may be saturated with active ingredient, and possibly a part of the active ingredient is adsorbed onto the inner surface of the particles (i.e. the surface surrounding the space or spaces in which the gas phase is present).

The invention particularly provides particles from which the volatile active ingredient, if desired, is released fast and suddenly upon opening of the enveloping phase or at least its becoming permeable to the active ingredient. Such a release is sometimes referred to as a “burst effect”. In this way, it is possible to realize for instance a process taste or odor, i.e. a taste or odor sensation resulting from a particular process step, for instance upon dissolution of the particles in a liquid, or upon heating of the particles, to a temperature at which the particles melt, a temperature at which decomposition of the particles occurs, or a temperature near or above the glass transition temperature.

Also envisaged is that through the invention extra stability of an active ingredient, in particular a (process) flavor or (process) aroma, can be realized. The fact is that frequently a moderate taste or odor stability has been described of products provided with, in particular, complex taste or odor systems such as process flavors or process aromas, as a result of undesired interactions with for instance the product matrix (see: Trend in Food Science & Technology 17 2006 236-243, K. B. DeRoos).

As particles according to the invention do not need to contain liquid carrier material (with active ingredient dissolved therein), also the risk of adverse visual effects resulting from such liquid phase (such as an oil film on a liquid in which the particles are dissolved) can be prevented. Thus, in a particular embodiment, the particles according to the invention are free of a liquid oil phase, more particularly free of a liquid phase. Possibly, the particles may contain a liquid phase, more specifically, a part of the active ingredient may have condensed to a liquid phase. Also if a part of the active ingredient is condensed, usually at least 50 wt. % of the active ingredient will be present in the gas phase. When a part of the active ingredient is condensed, the condensate will usually be present in one or more spaces in which also gaseous active ingredient is present.

The invention furthermore makes it possible to provide existing (hollow or porous) particles with a volatile active ingredient. This makes it for instance possible to stock up a large supply of particles as a basic material or to manufacture same in a single charge, if desired to store this charge for some time, and, shortly before sale, further processing into another product or final use, to provide the particles with volatile active ingredient. In this way, a single charge of basic material can be used for diverse applications, each with different active ingredients, which can have logistic advantages because, for instance, no (large amounts of) diverse end products (i.e. products with active ingredient) need to be kept in stock to enable rapid supply, since the active ingredient is not added until after the formation of the particles.

By the term ‘particle’ a material is meant which contains a conglomerate of molecules which at room temperature does not change in shape, or does so only very slowly, when it is placed freely on a surface. The particles contain one or more spaces, such as one or more cavities or pores, for holding a gas phase, which are at least substantially surrounded by an enveloping material, which is usually, at least at room temperature (20° C.), in a solid, understood to include amorphous, state. Usually, on average at least about 20% of the volume of the particles is constituted by space for holding a gas phase. Preferably, the total volume of the spaces is at least 30 vol. %, more preferably at least 40 vol. %. The total volume of the spaces usually covers 70 vol. % at a maximum, preferably 60 vol. % at a maximum. About 80 vol. % at a maximum of the particles is usually formed by a solid (enveloping) phase, preferably 70 vol. % at a maximum, more preferably 60 vol. % at a maximum. Usually the solid (enveloping) phase forms at least 30 vol. %, in particular at least 40 vol. %. The percentage of empty space in the particles can for instance be determined by measuring the density of the particles with helium pycnometry before and after milling.

In a preferred embodiment, the particles have been spray-dried, for instance in an otherwise known manner whereby a liquid mixture of the particle-forming material is mixed with gas and then spray-dried. The particles may in particular be microparticles, i.e. particles having a surface average particle size, as determinable by microscopy, or possibly by light scattering in for instance a Coulter counter, in the range of 1-1,000 μm.

Preferably, the surface average particle size is 250 μm at a maximum, in particular 200 μm at a maximum or 150 μm at a maximum. Preferably, the surface average particle size is 20 μm at a minimum, in particular 50 μm at a minimum or 70 μm at a minimum. Preferably, the particles form a powder.

A material is particularly deemed impermeable to an active ingredient if the active ingredient included in the particles remains at least substantially entrapped in the particles for a storage period of at least two months, preferably at least three months, more preferably at least six months, at storage temperature, for instance 20° C. or lower. What can be used as a guideline is that a material is at least impermeable to an active ingredient if the diffusion coefficient at the storage temperature, for instance about 20° C., is 10⁻¹⁴ m²s⁻¹ or less.

A material is particularly deemed permeable if the material, for instance at the temperature prevailing at mixing with the active ingredient, can diffuse through the material within about a day, preferably within three hours, more preferably within one hour, so that an equilibrium is achieved or at least approximated between the gas phase in the spaces of the particles and the gas phase surrounding the particles. What can be used as a guideline is that a material is at least permeable to an active ingredient if the diffusion coefficient at the storage temperature, for instance about 20° C., is 10⁻¹² m²s⁻¹ or more.

Eminently suitable as an enveloping phase are (amorphous) materials that have a glass transition temperature above the storage temperature of the particles, for instance a glass transition temperature of more than 25° C., at least 50° C., or at least 70° C. and which at a temperature below the glass transition temperature, for instance at a temperature of 50° C. or lower, preferably at a temperature of 25° C. or lower, are at least essentially impermeable to the active ingredient. The glass transition temperature is preferably 120° C. at a maximum or 100° C. at a maximum.

The permeability of the enveloping phase can usually be increased sufficiently by heating the particles to a temperature near or above the glass transition temperature, preferably at least 1° C., at least 5° C. or at least 10° C. above the glass transition temperature. With a view to the preservation of the particles and/or the prevention of unwanted organoleptic side effects, the temperature during the loading of the particles with active ingredient is usually 50° C. at a maximum above the glass transition temperature, preferably 25° C. or 15° C. at a maximum, with the proviso that the temperature as a rule is chosen so low that the particles at least substantially retain their form, i.e. below the melting or decomposition temperature of the enveloping phase.

The glass transition temperature, as used herein, can be determined by a DSC method described in Schoonman et al. (Biotechnology Progress 18 (2002) 139), in which an indium-calibrated TA8000/DSC 820 (Mettler-Toledo, Switzerland) is used, and in which the results are recorded and analyzed with the Mettler-GraphWare TA72.2/5 software package. For the DSC measurements, 25 mg of sample material is introduced into a hermetically sealed crucible, after which a first cycle is carried out with a heating rate of 5° C./min and a cooling rate of 20° C./min, followed by a second heating step with a heating rate of 5° C./min. The glass transition temperature is the prevailing temperature at onset of the change in the heat flow in the second heating curve.

On the basis of what is described herein, common general knowledge in the art and possibly some routine experimentation the skilled person will be able to choose suitable materials and conditions. Suitable materials can for instance include one or more substances selected from the group of carbohydrates, proteins and emulsifiers. One or more carbohydrates can for instance be chosen from sugars, maltodextrins and polysaccharides, such as starch. One or more proteins can optionally be selected from the group of caseinates, casein included, and whey proteins. An emulsifier will as a rule be present in a relatively small content, for instance 10 wt. % at a maximum, based on dry matter.

Optionally one or more glass transition temperature modifying additives may be added, such as one or more softeners, for instance moisture. Further, the glass transition temperature may depend on the degree of polymerization. As a rule, the glass transition temperature of a material is higher according as the average molecular size of the material is higher.

In an embodiment, gas is mixed with the particles. This gas can then comprise an active ingredient, a precursor for the active ingredient and/or a carrier gas. The carrier gas can be selected in particular from the group of nitrogen, carbon dioxide, di-nitrogen oxide (laughing gas), oxygen, noble gases, including mixtures of two or more of these carrier gases. If a carrier gas is used, as a rule the preparation conditions are chosen such that the carrier gas in the eventual particles is not condensed or otherwise liquefied. At least if particles are provided with an active ingredient which is sensitive to oxidation, during preparation usually use is made of an oxygen-free carrier gas, preferably a carrier gas at least essentially consisting of one or more gases selected from nitrogen, carbon dioxide, laughing gas and noble gases, optionally mixed with an active ingredient (precursors for active ingredients included).

Examples of such oxidation-sensitive active ingredients are volatile fatty acids, in particular polyunsaturated fatty acids, such as omega-3 fatty acids (alpha-linolenic acid, eicosapentaenoic acid, docosahexaenoic acid).

The particles can be free of carrier gas or contain a carrier gas. If carrier gas is present, the ratio of carrier gas to active ingredient can be selected within wide ratios, for instance a weight ratio of at least 0.01, at least 0.05 or at least 0.1. The weight ratio can for instance be 10 at a maximum.

Mixing can be done under atmospheric pressure or at elevated pressure, for instance at a pressure of 50 bara at a maximum or 40 bara at a maximum. In an embodiment, the pressure is 2 bara at a maximum, 5 bara at a maximum or 10 bara at a maximum. Through the invention it is possible to obtain particles of which the gas phase in the one or more spaces (at 25° C.) has a pressure of less than 1 bara, approximately 1 bara or more than 1 bara, as in the range of 0.5-50 bara, of 1-40 bara or of 2-10 bara.

Mixing is generally continued at a temperature at which the enveloping material is permeable to the active ingredient until the space(s) in the particles is (/are) sufficiently provided with active ingredient. Usually this step is carried out for 24 hours at a maximum, preferably for 6 hours at a maximum, more preferably for 2 hours at a maximum or 1 hour at a maximum. Usually, this step is carried out for 1 min. at a minimum, preferably for 15 min. at a minimum, more preferably for 30 min. at a minimum.

The process is suitable for preparing particles with diverse (volatile) active ingredients. In particular, the active ingredient can be selected from the group of flavors and aromas, which preferably have a measurable vapor pressure at room temperature. The aroma can for instance be chosen from the group of fruit aromas, coffee aromas, herb aromas, tea aromas, milk aromas, meat aromas, cheese aromas, butter aromas, cream aromas, flower aromas, tree aromas, etc. Such aromas are well known to the skilled person, for instance from http://en.Wikipedia.org under the entry “ester” or “aroma compound”. In particular, the active ingredient can be an aromatic ester, aldehyde, amine, alcohol, ether, ketone, terpene or thiol. As intended herein, an aromatic compound is a compound with an aroma, such as an ester of acetate, butyrate, valerate, hexanoate, heptanoate, octanoate, nonanoate, decanoate, undecanoate or laurate, while the alcohol residue can be, for instance, allyl, benzyl, bornyl, ethyl, geranoyl, isopropyl or isobutyl. Other aromas are for instance aromas mentioned in the above-cited prior art, whose contents regarding the aromas mentioned is incorporated herein by reference.

In an embodiment, particles are prepared with an active ingredient which during heating is converted into the active ingredient desired for the end product (i.e. the particles according to the invention or a product, such as a consumer product, in particular a foodstuff, which contains the particles). Such a substance to be converted is also referred to as precursor. Such precursors can be selected in particular from the group of flavor precursors and aroma precursors. The precursor can be a volatile compound or a compound solid or liquid at ambient temperature which reacts during heating to form the volatile active ingredient, such as an aroma or flavor.

A flavor precursor and/or aroma precursor can optionally be used in combination with another (volatile) active ingredient for the preparation of the particles.

If desired, one or more precursors can be incorporated in the particles during a process according to the invention. It is also possible for one or more precursors to be incorporated into the particles beforehand, during the manufacture of particles intended to be provided with a gaseous active ingredient. The precursor can for instance be mixed with the material with which the particles are manufactured, in particular with the aid of spray-drying.

The precursor may be converted during filling of the space(s) in the particles, provided that this is done at a suitable temperature for that purpose. In an embodiment, the precursor is converted in a later stage, when further processing the particles, or in the preparation of use for consumption, for instance when heating a foodstuff, beverages included, for consumption.

Typical flavor precursors or aroma precursors, as is known, can give rise via (a dry phase) Maillard reaction to specific tastes or odors such as the taste or odor of ‘popcorn’, or ‘roasty odors’ which may be desirable especially in bakery, savory or confectionery products. Typical precursors for this are amino acids, hydrolyzed proteins, reducing carbohydrates and the like. Examples of suitable amino acids are beta-alanine and proline. Examples of suitable carbohydrates are fructose, glucose or maltose (see for instance J. Agric. Food Chem. 1998, 46, 2721-26, T. Hofmann and P. Schieberle; J. Agric. Food Chem. 1994, 42, 1080-1084, S. Nishibori and S. Kawakishi). Obtaining a Maillard taste or odor via such precursors is known to the skilled person: typical reaction conditions in dry systems are temperatures of 100-160° C., more specifically 110-140° C., usually with reaction times of 2-30 minutes, more specifically of 5-15 minutes.

The content of active ingredient, in particular aroma, flavor, or a precursor for an aroma or flavor, in the product obtained by the invention is usually at least 0.005 wt. % based on the weight of the particles, preferably at least 0.01 wt. %, more particularly at least 0.05 wt. % or at least 0.1 wt. %. The upper limit partly depends on the vapor pressure at the mixing temperature, the diffusion rate and the duration of mixing prior to cooling. The content of active ingredient, in particular aroma, in the product obtained by the invention is usually 2 wt. % at a maximum, based on the weight of the particle, in particular 1 wt. % at a maximum or 0.5 wt. % at a maximum.

If desired, the particles may be provided with active ingredient in the presence of an anticlotting agent or a free flowability agent to prevent excessive aggregation of the particles, especially during a heating step. Such agents are known per se, for instance silicon oxide particles. These can be mixed with (carrier) gas and the particles which are provided with active ingredient.

A process according to the invention is particularly suitable for preparing a foodstuff or foodstuff ingredient, preferably a foodstuff or a foodstuff ingredient chosen from the group of baking mixes, chips, savory snacks, appetizers, pre-fried pasta, such as pre-fried bread or pastry, seasoning, marinades and instant products such as instant soups, instant sauces and instant beverages, such as instant soft drinks, instant thirst-quenchers, instant energy drinks, instant coffee, instant tea, and the like.

Furthermore, a process according to the invention is suitable for preparing a personal care product or a household product, preferably a personal care product or household product chosen from the group of cosmetics, perfumes, creams, deodorants, soaps for personal care and soaps with household application, such as for instance detergents, washing-up agents, dishwashing agents, polishing waxes.

The invention will now be elucidated in and by a few examples.

### EXAMPLES

### Manufacture of Particles

By means of spray-drying, in an otherwise conventional manner, from a solution of maltodextrin and emulsifying starch (46% maltodextrin, 4% emulsifying starch, balance water) with injection of nitrogen gas on the product line, a foamed powder was produced. This powder was treated in the following manner.

### Example 1

A pressure vessel was filled with 20 kg of powder. Eight grams of a mixture consisting of 10 different aromas (ethyl esters of C₂-C₁₂ carboxylic acids) were mixed with 600 grams of silicon dioxide (sipernat). This mixture was added to the powder. Next, the vessel was set at a pressure of 35 bar using nitrogen, and the powder, while kept in motion, was heated to 140° C. and held at this temperature for 1 hour. This was followed by cooling to 30° C. and letting off the pressure.

### Example 2

Example 1 was repeated with the proviso that the vessel was first adjusted to 35 bar, then the pressure was let off and the aroma/sipernat mixture was added, after which the vessel was adjusted to a pressure of 5 bar, and then heated, etc.

### Example 3

Example 1 was repeated except that 40 grams of aroma mixture were used.

### Results

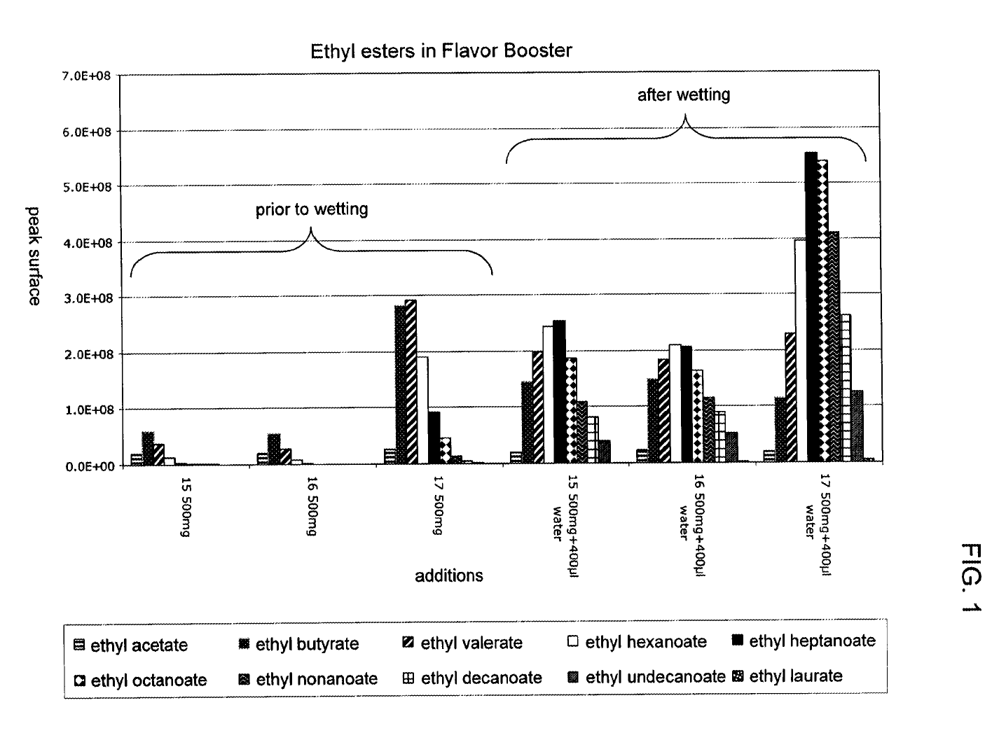

The powders were analyzed as follows: 0.5 gram of the powders was added to a small pot having a contents of 10 ml. After waiting for some time, with the aid of a GCMS it was analyzed what components were present in the gas phase. The same experiment was done with 0.4 ml of water being injected into the pot. FIG. 1 shows that dissolving the powder leads to the increased release of aromas.

1-24. (canceled)

25. A process for preparing particles, which particles contain one or more spaces in which a gas phase is present which comprises at least one active ingredient, in particular at least one aroma, flavor or precursor for an aroma or flavor, and which space or spaces are at least substantially surrounded by an enveloping phase which at ambient temperature is at least substantially solid and at least substantially impermeable to the active ingredient, comprising allowing the gaseous active ingredient to migrate from or through the enveloping phase into the space or spaces at a temperature at which the enveloping phase is permeable to the gaseous active ingredient; and then cooling the particles to a temperature at which the enveloping phase of the particles is at least substantially impermeable to the active ingredient in the particles.

26. A process according to claim 25, comprising mixing the particles, which contain one or more spaces, with a gas which comprises gaseous active ingredient, at a temperature at which the enveloping phase is permeable to the active ingredient, whereby gaseous active ingredient migrates into the spaces of the particles; and then cooling the particles to a temperature at which the enveloping phase of the particles is at least substantially impermeable to the active ingredient in the particles.

27. A process according to claim 25, wherein the enveloping phase comprises a precursor for the active ingredient, which precursor upon heating is converted into the gaseous active ingredient, comprising heating the particles of which the enveloping phase contains a precursor, thereby forming the gaseous active ingredient; allowing the gaseous active ingredient to migrate from the enveloping phase into the space or spaces; and then cooling the particles to a temperature at which the enveloping phase of the particles is at least substantially impermeable to the active ingredient, wherein preferably the particles are heated while mixing the particles with a gas.

28. A process according to claim 25, wherein the particles and the gas are mixed at a temperature above the glass transition temperature of the enveloping phase.

29. A process according to claim 25, wherein the enveloping phase comprises at least one substance selected from the group of carbohydrates, proteins and emulsifiers, in particular from the group of maltodextrins, starch and other polysaccharides, sugars, caseinate and whey protein.

30. A process according to claim 25, wherein the particles are a foodstuff or a foodstuff ingredient, preferably a foodstuff or a foodstuff ingredient selected from the group of baking mixes, chips, savory snacks, appetizers, seasoning, marinades and instant products, such as instant coffee, instant tea, instant soups and instant sauces and instant beverages or wherein the particles form a personal care product, household product, an ingredient for a personal care product or an ingredient for a household product, preferably a personal care product or household product selected from the group of cosmetics, perfumes, creams, deodorants, soaps for personal care and soaps with household application, such as for instance detergents, washing-up agents, dishwashing agents, polishing waxes.

31. A process according to claim 25, wherein the active ingredient is selected from the group of aromas and flavors, preferably an aroma selected from the group of aromatic esters (for instance ethyl esters), aldehydes, amines, alcohols, ethers, ketones, terpenes and thiols.

32. A process according to claim 25, wherein the spaces on average comprise 10 to 70 vol. %, preferably 40 to 60 vol. %, of the particles.

33. A process according to claim 25, wherein the particles are provided with 0.01-1 wt. % of active ingredient based on the total weight of the particles.

34. A process according to claim 25, wherein the cooled particles contain 0.4 to 8 wt. %, based on the total weight of the particles, of gas.

35. Particles obtainable by a process according to claim 25, wherein preferably at least the enveloping phase is at least essentially free of a liquid phase.

36. Particles according to claim 35, which particles are free of a liquid phase other than a liquid phase formed from a part of the active ingredient, or which particles are wholly free of a liquid phase.

37. Particles according to claim 35, wherein 25 wt. % or less of the active ingredient present in the particles, is present in the enveloping material.

38. Spray-dried powder, which powder comprises particles, which particles contain one or more spaces in which a gas phase is present which comprises at least one gaseous active ingredient, preferably an active ingredient selected from the group of aromas, flavors, aroma precursors, flavor precursors, and oxidation-sensitive active ingredients.

39. A foodstuff, foodstuff ingredient, personal care product, household product, an ingredient for a personal care product or an ingredient for a household product comprising particles according to claim 35.

40. A foodstuff, foodstuff ingredient, personal care product, household product, an ingredient for a personal care product or an ingredient for a household product comprising a powder according to claim 35.
